(19) United States
(12) Patent Application Publication
(10) Pub. No.: US 2010/0298418 A1
(43) Pub. Date: Nov. 25, 2010

(54) Assay for measuring plasma FGL-2 and methods and uses thereof

(75) Inventor: Gary Levy, Thornhill (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: Veritas Therapeutics Inc., Thornhill (CA)

(21) Appl. No.: 12/784,006

(22) Filed: May 20, 2010

Related U.S. Application Data
(60) Provisional application No. 61/179,993, filed on May 20, 2009.

(51) Int. Cl. C07K 16/18; G01N 33/53; C12Q 1/70; A61K 31/7105; A61P 31/12

(52) U.S. Cl. 514/44 R; 435/7.92; 435/5; 530/387.1

(57) ABSTRACT

The disclosure relates to methods for identifying a subject having elevated FGL2 plasma levels, the method comprising the step of assaying a plasma sample from the subject to determine a level of FGL2, wherein a level above control indicates the subject has elevated FGL2 levels. Diagnostic methods, methods of monitoring prognosis and methods of medical treatment relating to viral hepatitis are also provided. The disclosure also provides assays and kits useful in the diagnosis of a viral hepatitis associated with elevated FGL2.

### RELATED APPLICATIONS

This application claims the benefit under 35 USC §119(e) of U.S. provisional application No. 61/179,993 filed May 20, 2009, which is incorporated herein in its entirety.

### FIELD

The disclosure relates to an assay for detecting and measuring plasma levels of FGL2/fibroleukin found in plasma as well as assays useful for diagnosing and monitoring medical conditions associated with elevated levels of FGL2, such as viral hepatitis.

### BACKGROUND

Recently, a population of naturally occurring CD4⁺CD25⁺ regulatory T (Treg) cells has been identified with an important role in the maintenance of peripheral self-tolerance (Sakaguchi 2005). Depletion or functional alteration of this subset in normal animals results in the development of autoimmune diseases (Sakaguchi 2005). In recent years, there has been an intensive investigation to define the effector molecules that account for the immunosuppressive activity of Treg cells, which may be targets for the development of novel therapeutic strategies for diseases in which alterations in Treg activity may play a pathogenic role including chronic viral hepatitis B and C, cancer and autoimmune diseases including rheumatoid arthritis, inflammatory bowel disease and diabetes mellitus (Sakaguchi et al. 1995; Bach and Chatenoud 2001; Wood and Sakaguchi 2003; Cabrera et al. 2004).

Treg have now been implicated in suppressing the T and B cell immune responses to viral infections including hepatitis B (HBV) (Stoop et al. 2005; Xu et al. 2006) and hepatitis C(HCV) (Boettler et al. 2005; Rushbrook et al. 2005). Patients with chronic HBV and HCV infection have increased numbers of Tregs both in the blood and within the liver which have been reported to impair immune responsiveness to these viruses (Cabrera et al. 2004; Xu et al. 2006). In support for a role of Treg in the pathogenesis of HBV is the observation that depletion of Treg in a mouse model of chronic HBV results in enhanced anti viral CD8⁺ T cell immunity (Furuichi et al. 2005).

Fibrinogen-like protein 2 (fgl2) has been classified as a member of the fibrinogen-like family of proteins which include tenascin and angiopoietin (Doolittle 1983). Previously a role for FGL2 was reported in innate immunity, expressed by macrophages and endothelial cells as a membrane associated prothrombinase accounting for the fibrin deposition seen in both acute and chronic hepatitis in mice and humans (Levy et al. 1981; Levy et al. 2000; Marsden et al. 2003). Fibrinogen-like protein 2 (fgl2) has been recently identified by microarray analysis screening as a putative candidate gene for Treg cell function (Herman et al. 2004; Fontenot et al. 2005). Herman et al. were the first to report an increased expression of fgl2 in CD4⁺CD25⁺Treg cells in an insulitis model (Herman et al. 2004). Following this report, Rudensky et al. showed high levels of fgl2 transcripts in freshly isolated Foxp3⁺CD4⁺Treg cells (Fontenot et al. 2005; Williams and Rudensky 2007). These findings are consistent with earlier studies by Marazzi et al. (Marazzi et al. 1998), who demonstrated preferential expression of fgl2 in CD45R0⁺ memory T cells; in both animals and humans, circulating Treg cells have been consistently defined as belonging to the memory T cell compartment.

In vitro studies by Chan et al. have provided the first evidence that FGL2 has immunomodulatory activity (Chan et al. 2003). Recombinant FGL2, which was generated in a baculovirus expression system, inhibited T cell proliferation in response to alloantigens, anti-CD3/anti-CD28 monoclonal antibody and Con A in a dose-dependent manner, whereas it had no direct inhibitory effect on CTL activity (Chan et al. 2003). The inhibitory effect of FGL2 was prevented by the use of anti-FGL2 antibody. Addition of FGL2 to allogeneic cultures caused the polarization of the immune response towards a Th2 cytokine profile with increased levels of IL-4 and IL-10 and decreased levels of IL-12 and IFN-γ (Chan et al. 2003). In addition, FGL2 abrogated the LPS-induced maturation of BM-derived dendritic cells (DC) by inhibiting NF-κB nuclear translocation, resulting in reduced expression of CD80 and MHCII and impaired induction of alloreactive T cell proliferation. The immunosuppressive activity of FGL2 was localized to the C-terminal region which contains the fibrinogen related domain (FRED) (Chan et al. 2003).

The present inventors previously reported a high level of fgl2 mRNA expression in Treg cells by real-time PCR. They showed a statistically significant increase in the percentage of Treg cells in all lymphoid tissues of fgl2^{-/-} mice compared to the wild type (fgl2^{+/+} controls). Similarly, Foxp3 expression levels were also increased in Treg cells from fgl2^{-/-} mice compared with wild type controls, however, the suppressive activity of Treg cells isolated from fgl2^{-/-} mice was significantly impaired, and antibody to FGL2 completely inhibited wild-type Treg cell activity in vitro (Shalev et al. 2009). Consistent with the importance of FGL2 to Treg cell function, targeted deletion of fgl2 resulted in increased immune reactivity of T, B and DC cells and glomerulonephritis in aged fgl2^{-/-} mice (Shalev et al. 2008). Moreover, T cells from fgl2^{-/-} mice showed greater proliferation in response to Con A and alloantigens, as well as Th1 cytokine-polarized immune responses. Increased antibody-producing B cells were observed in fgl2^{-/-} mice following LPS and NP-Ficoll stimulation. DC from fgl2^{-/-} mice showed higher expression of CD80 and MHCII, and an increased migration rate into the periarteriolar lymphoid sheath (PALS) following stimulation. Six-month-old fgl2^{-/-} mice were smaller in size and developed autoimmune glomerulonephritis characterized by deposits of immunoglobulins (IgG and IgM), infiltrates of macrophages and lymphocytes, fibrin deposition, haemorrhage and glomerulosclerosis and tubular atrophy. Fgl2^{-/-} mice had normal proportions of T cells, B cells and macrophages, but there was an increase in DC possibly due to a decrease in apoptosis (Shalev et al. 2008). These results are consistent with those found by Hancock et al., who showed that fgl2^{-/-} mice have an intact Th1 immune response (Hancock et al. 2004).

Foxp3⁺ CD4⁺CD25⁺ natural Treg cells (Treg) are known to be actively engaged in the negative control of a variety of physiological and pathological immune responses (Sakaguchi 2005). Evidence that Tregs may underlie the attenuated HCV specific T cell responses observed in chronically infected patients comes from the finding that Tregs isolated from the peripheral blood of HCV patients suppress HCV specific responses in-vitro (Boettler et al. 2005; Rushbrook et al. 2005). Furthermore, Tregs are elevated in the circulation of HCV patients and not in those who clear the virus (Cabrera et al. 2004). A number of other studies have suggested that depletion or reduction of Treg cells leads to enhanced immune responses against various infectious pathogens including HBV and HCV (Cabrera et al. 2004; Xu et al. 2006; Belkaid and Rouse 2005). Thus, Treg cells appear to suppress the effective response of virus-specific T cells in patients with HCV. The suppressive activity of Tregs has been suggested to be mediated through cytokines including TGF-β and IL-10 (Miyara and Sakaguchi 2007). The present inventors and others have recently demonstrated that FGL2, a member of the fibrinogen-related superfamily of protein is highly expressed by Tregs and contributes to their suppressive activity (Shalev et al. 2009).

### SUMMARY

It has now been shown that FGL2 is detectable in plasma and its levels correlate with the severity of viral hepatitis. Accordingly, in one aspect, the present disclosure provides a method for identifying a subject having an elevated FGL2 level in plasma, comprising the steps of assaying a plasma sample from the subject to determine the level of FGL2, wherein a level above control indicates that the subject has elevated FGL2 levels. In one embodiment, the method comprises the steps of (a) providing a plasma sample from the subject; and (b) determining the level of FGL2 in the sample, wherein a FGL2 level above control indicates the subject has or is at risk for said viral hepatitis.

Also provided herein is a method for monitoring disease progression of viral hepatitis associated with elevated FGL2 in plasma of a subject, the method comprising the steps of:

a) at a first time point, determining the level of FGL2 in a plasma sample from the subject; and

b) comparing the level of FGL2 in a plasma sample taken from the subject at a second time point different from the first time point; wherein a difference in the FGL2 levels at the first time point compared to the second time point indicates modulated progression of the viral hepatitis. In one embodiment, the difference in the FGL2 levels is an increase.

Also provided herein is a method of monitoring the response to anti-viral therapy in a subject infected with viral hepatitis, the method comprising the steps of:

a) at a first time point, determining the level of FGL2 in a plasma sample from the subject; and

b) comparing the level of FGL2 in a plasma sample taken from said subject at a second time point different from said first time point; wherein a decrease in the FGL2 levels at the second time point compared to the first time point indicates response to anti-viral therapy.

Further provided is a method for predicting the response to anti-viral therapy and clearance of the virus in a subject infected with viral hepatitis, the method comprising the steps:

a) determining the level of FGL2 in a plasma sample from the subject prior to initiation of antiviral therapy;

b) comparing the level in a) to a control;

wherein a FGL2 level above control indicates non response to antiviral therapy.

Further, there is provided a method of genotyping a subject infected with HCV comprising the steps:

a) assaying plasma from the subject to determine a level of FGL2; and

b) comparing to a reference level; wherein similarity with the reference level indicates the genotype.

In another aspect, the disclosure also provides a method of treating or controlling progression of a viral hepatitis associated with elevated FGL2 levels in plasma, comprising the steps of identifying a subject having elevated FGL2 as determined by the methods described herein, and treating the subject with an agent that inhibits FGL2.

In another embodiment, the methods of the disclosure are used to determine the prognosis in a subject with viral hepatitis, comprising the steps of: assaying plasma from the subject to determine a level of FGL2; and comparing to a reference level; wherein a level above the reference level is indicative of poor prognosis.

In yet another aspect, the disclosure provides an assay useful in the diagnosis of a viral hepatitis comprising the steps of:

a) providing a plasma sample from a subject;

b) reacting the sample with an antibody that binds FGL2;

c) detecting bound FGL2; and

d) comparing the level of FGL2 in the sample with the level of FGL2 in a control subject, wherein a subject having said viral hepatitis is indicated by a greater level of FGL2 in the sample relative to the level of FGL2 in the control subject. In one embodiment, the assay is an enzyme-linked immunosorbant assay (ELISA).

In a further aspect, the disclosure provides a kit comprising an antibody that binds FGL2 and instructions for the use thereof in determining the level of FGL2 in a plasma sample.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the mouse and human FGL2 ELISA standard curve: The sensitivity of the ELISA was assessed by measuring various concentration of FGL2. The assay shows a linear regression between absorbance at 450 nm and concentration of FGL2 (R²=0.99 for both mouse and human assay). The detection range was between 5 ng/mL and 500 ng/mL for mouse ELISA (FIG. 1a) and 0.3 ng/ml and 125 ng/ml for human (FIG. 1b).

FIG. 2 shows the level of plasma FGL2 at baseline and after MHV-3 infection in Mice. Resistant (A/J) and susceptible (Balb/cJ) mice were infected with 100 PFU of MHV-3 at day 0. Animals (n=3/group/timepoint) were sacrificed and blood collected and analyzed for levels of FGL2 by ELISA prior to infection (day 0) and at different timepoints post-infection. Data represent mean±SD. A two way ANOVA was used to compare means, *p<0.001

FIG. 3 shows the mean plasma levels of FGL2 in healthy controls according to gender and ethnicity. Ten ml of heparinized blood was collected from healthy controls (n=30). The blood is centrifuged at 1500×g for 10 minutes at 21° C. and the plasma is collected. Plasma diluted 1/10 with 2.5% BSA was analyzed for presence of FGL2. Levels of FGL2 did not differ significantly according to gender (3A) (male vs. female: 38.8±23.4 ng/mL vs. 34.2±22.0 ng/mL, p=0.5), or ethnicity (3B) (Caucasian vs. Asian: 37.9±22.9 ng/mL vs. 35.3±22.4 ng/mL, p=0.2).

FIG. 4 shows the mean plasma levels of FGL2 in patients with chronic HCV infection. A) Ten ml of heparinized blood was collected from 80 patients with chronic HCV infection, which had not received anti-viral therapy. Mean plasma levels of FGL2 in these patients were compared to 30 healthy controls, 24 patients with inactive alcoholic cirrhosis and 32 patients with chronic HCV who cleared the virus following successful anti-viral therapy (sustained virological responders, SVR). Mean plasma levels of FGL2 were significantly higher in patients with chronic HCV infection ((84.3±89.1 ng/mL, n=80) compared to healthy controls (36.41±21.9 ng/mL, n=30, p<0.001), patients with alcoholic cirrhosis (18.8±17.4 ng/mL, n=24, p<0.001), and patients with. SVR (16.6±19.7 ng/ml, n=32, p<0.001). B) Among HCV patients, mean plasma levels of FGL2 were significantly higher in patients with cirrhosis (164.1±121.8 ng/mL, n=20) compared to non cirrhotics (57.7±52.8 ng/mL, n=60, p=0.001).

FIG. 5 shows the correlation between FGL2 levels and levels of aspartate transaminase (AST), alanine transaminase (ALT), coagulation (INR), and bilirubin. A positive correlation was found between plasma levels of FGL2 and AST, ALT (5A), coagulation (INR) (5B) and bilirubin (5C) whereas a negative correlation was observed between levels of FGL2 and albumin (5D).

FIG. 6 shows the mean plasma levels of FGL2 in patients with chronic HCV infection according to the stage of fibrosis. 6A) Plasma levels of FGL2 were significantly higher in HCV patients with advanced fibrosis (stage 3-4, n=26) compared to the patients with lower stage of fibrosis (148.3+143.0 vs. 44.4+52.4 ng/ml for stage≦1, n=33, p=0.001) and (148.3+143.0 vs. 72.9+65.9 ng/ml for stage 2, n=12, p=0.01). 6B) Plasma levels of FGL2 is also significantly higher in patients with higher activity grade (grade≧2, n=26), compared to those with lower activity grade (grade<2, n=46), (74.4+69.8 vs. 36.8+37.1 ng/ml, p=0.01).

FIG. 7 shows the mean plasma levels of FGL2 in patients with chronic HCV infection according to genotype. 7A) There was no significant correlation between plasma levels of FGL2 and the viral titers. 7B) Plasma levels of FGL2 were significantly higher in patients with genotype 1 compared to genotype 2/3 infection (12.4±12.3 vs. 5.5±5.1 ng/ml, p=0.008).

FIG. 8 shows an FGL2 time course in two patients with chronic HCV infection treated with antiviral therapy. A) Patient 1 with genotype 1 infection did not respond to 48 weeks of therapy with pegylated interferon and Ribavirin. Plasma FGL2 levels in this patient were high prior to initiation of therapy, throughout treatment and 6 months after completion of therapy. B) Patient 2 with genotype 2 infection responded to antiviral therapy. Plasma levels of FGL2 were low prior to initiation of treatment and fell within 4 weeks of therapy to levels similar to healthy controls and were undetectable after completion of therapy.

FIG. 9 shows FGL2 and FoxP3 expression in the liver of patients with chronic HCV infection. Panel A: Staining for FGL2. Heavy, predominantly lymphocytic infiltrates within the portal and periportal areas of the liver. Note that many infiltrating cells stain positively for FGL2 (brown stain) which is expressed both within the cytoplasm and at the cell surface (arrows). Panel B: FoxP3 staining. Similar infiltrates as in panel A, but showing nuclear FoxP3 reactivity (brown stain, arrows). Panel C: Co-localization of FoxP3 and FGL2 is seen in some but not all cells staining with either FoxP3 (Tetrazolium blue, nuclear) or FGL2 (brown, cytoplasmic/membranous). Panel D: Negative control for FGL2. Absence of cytoplasmic staining validates the specificity of FGL2 reactivity in panels A and C (original magnification, Panels A-D=×200).

FIG. 10 shows liver histology and expression of FGL2 in two patients with chronic HCV infection. A. Patient 1 has severe chronic active hepatitis with many FGL2 positive cells (arrows). B. Patient 2 has histologic evidence of chronic active hepatitis with a few scattered FGL2 positive cells (arrows). Staining for FGL2 was by standard immunohistochemistry using a monoclonal anti-FGL2 and horse radish peroxidase. ×300.

### DETAILED DESCRIPTION

The present inventors have shown that plasma levels of FGL2 were higher in susceptible BALB/cJ mice with MHV-3-induced hepatitis than in A/J mice known to be resistant to MHV-3. Similarly, plasma FGL2 levels were higher in chronic hepatitis C patients than in healthy controls. Liver biopsy expression of FGL2 in hepatic lymphocytic cell infiltrates in hepatitis C by immunohistochemistry was increased and correlated with the plasma FGL2 levels. These data provide evidence that measurement of FGL2 may serve as a useful biomarker for predicting HCV disease activity and response to anti-viral treatment.

The methods described herein are useful for detecting increases and decreases in blood FGL2 levels. In one embodiment, plasma or serum FGL2 levels are measured.

In one aspect, the disclosure provides a method for determining the level of FGL2 in plasma, the method comprising contacting a plasma sample from a subject with an agent that specifically binds FGL2 and detecting the binding thereof to determine the level of FGL2.

The term “FGL2 protein” or “fibroleukin protein” or “fibrinogen-like 2 protein” as used herein refers to a non-membrane bound FGL2 protein detectable in plasma including soluble FGL2 from any species or source and includes analogs and fragments or portions of a soluble FGL2 protein.

The FGL2 protein may have any of the known published sequences for fgl2 which can be obtained from public sources such as GenBank. Examples of such sequences include, but are not limited to Accession Nos. AAL68855; P12804; Q14314; NP032039; AAG42269; AAD10825; AAB88815; AAB88814; NP006673; AAC16423; AAC16422; AAB92553. The fgl2 sequences can also be found in WO 98/51335 (published Nov. 19, 1998) and in Marazzi et al. (1998), Rüegg et al. (1995) and Yuwaraj et al. (2001)). The aforementioned sequences are incorporated herein by reference. The FGL2 protein can be obtained from any species, preferably a mammal including human and mouse.

FGL2 is considered to be “over-expressed”, “increased”, “upregulated” or “elevated” in a subject when levels of FGL2 in that subject exceed levels of FGL2 in a suitable control.

The term “subject” as used herein includes all members of the animal kingdom and is preferably a mammal, more preferably a human.

The term “viral hepatitis” refers to a disease caused by a virus that results in inflammation of the liver and includes, without limitation, hepatitis viruses A, B, C, D and E in humans, murine hepatitis virus strain 3 (MHV-3) and lymphocytic choriomenigitis virus (LCMV) in mice. In one embodiment, the viral hepatitis is mouse hepatitis virus strain 3. In another embodiment, the viral hepatitis is chronic hepatitis C (HCV). In yet another embodiment, the viral hepatitis is chronic hepatitis B (HBV).

In one embodiment, the method for identifying a subject having or at risk for a viral hepatitis associated with elevated FGL2 in plasma comprises the steps:

a) providing a plasma sample from said subject; and

b) determining a level of FGL2 in said sample,

wherein a FGL2 level above control indicates the subject has or is at risk for said viral hepatitis.

The term “control” and/or “suitable control” as used herein includes subject or subjects that are healthy and/or do not have viral hepatitis or increased risk of viral hepatitis, a sample obtained from, or a level derived from a subject or subjects that are healthy and/or do not have viral hepatitis or increased risk of viral hepatitis. For example, where the viral hepatitis is chronic hepatitis C(HCV), the control is a sample from a subject or subjects that do not have HCV associated with increased plasma FGL2, and/or is a value reflecting the level of plasma FGL2 in a subject or subjects that do not have viral hepatitis associated with increased FGL2. As used herein a “control subject” is a subject or group of subjects that are healthy and/or do not have viral hepatitis, a “control sample” is a sample derived from a control subject and a “control level” is a level of plasma FGL2 in a control sample or control subject.

The disclosure provides that the mean level of plasma FGL2 in human control subjects is 36.4±21.9 ng/mL. The mean FGL2 level in HCV patients was 84.3±89.1 ng/mL. The mean level of plasma FGL2 in mice resistant to MHV-3 i.e. mouse control subjects is 79+/−17 ng/ml. The mean level of plasma FGL2 in mice susceptible to MHV-3 is 124+/−36 ng/ml.

Accordingly, in one embodiment the control level is less than 50 ng/ml, 45 ng/ml, 40 ng/ml, 35 ng/ml, 30 ng/ml, 25 ng/ml, 20 ng/ml or 15 ng/ml for human. In one embodiment, the control level is from 30-40 ng/ml or about 30 ng/ml. In another embodiment, the control level is less than 60 ng/ml, 65 ng/ml, 70 ng/ml, 75 ng/ml, 80 ng/ml, 85 ng/ml or 90 ng/ml for mouse. In one embodiment, the control level is from 60-85 ng/mL.

In another embodiment, the level of plasma FGL2 in a human subject tested is greater than 60 ng/ml, 65 ng/ml, 70 ng/ml, 75 ng/ml, 80 ng/ml, 85 ng/ml or 90 ng/ml. In an embodiment, the level of plasma FGL2 in a human indicative of viral hepatitis or a risk of viral hepatitis is greater than 60 ng/mL.

In a further aspect, there is provided a method for monitoring progression of a viral hepatitis associated with elevated FGL2 in plasma of a subject, the method comprising the steps of:

a) at a first time point, determining the level of FGL2 in a plasma sample from the subject; and

b) comparing the level of FGL2 in a plasma sample taken from said subject at a second time point different from said first time point;

wherein a difference in the FGL2 levels at the first time point compared to the second time point indicates modulated progression of the viral hepatitis.

The phrase “a difference in the FGL2 levels at the first time point compared to the second time point” means having a difference that is 2 or more standard deviations above or below the mean of the plasma level of FGL2.

The present inventors have shown that those patients who responded to anti-viral therapy (SVR) had levels similar to those found in healthy controls whereas levels remained high in non-responders. Accordingly, there is provided a method of monitoring the response to anti-viral therapy in a subject infected with viral hepatitis, the method comprising the steps of:

a) at a first time point, determining the level of FGL2 in a plasma sample from the subject; and

b) comparing the level of FGL2 in a plasma sample taken from said subject at a second time point different from said first time point;

wherein a decrease in the FGL2 levels at the second time point compared to the first time point indicates response to anti-viral therapy. In one embodiment, the first time point precedes the start of anti-viral therapy.

In an embodiment, the method is repeated at a third, fourth, fifth or later time point to continually monitor anti-viral therapy. Such time points may be separated by 1 week or 1 month or more. Typically, blood is drawn pre-treatment, 1 week, 4 weeks, 12 weeks, 24 weeks and 48 weeks. The term “anti-viral therapy” as used herein includes, without limitation, ribavirin and interferon.

Also provided herein is a method for predicting the response to anti-viral therapy and clearance of the virus in a subject infected with viral hepatitis, the method comprising the steps:

a) determining the level of FGL2 in a plasma sample from the subject prior to initiation of antiviral therapy;

b) comparing the level in a) to a control;

wherein a FGL2 level above control indicates non response to antiviral therapy. In one embodiment, an FGL2 level above 60 ng/ml indicates non response to antiviral therapy.

In yet another aspect, there is provided a method for determining the prognosis in a subject with viral hepatitis, comprising the steps of:

a) assaying plasma from the subject to determine a level of FGL2; and

b) comparing to a reference level;

wherein a level above the reference level is indicative of poor prognosis.

The phrase “a level above the reference level” means having a level that is 0.5, 1, 2 or 3 standard deviations higher above the reference level.

The term “poor prognosis” as used herein refers to prognosis associated with disease forms that are more aggressive and/or less treatable. For example, aggressive less treatable forms have poorer survival than less aggressive and/or treatable forms.

Additionally, it was found that plasma FGL2 levels were higher in HCV genotype 1-infected patients than those with genotype 2/3. Accordingly, in an aspect, there is provided a method of genotyping a subject infected with HCV comprising the steps:

a) assaying plasma from the subject to determine a level of FGL2; and

b) comparing to a reference level;

wherein similarity with the reference level indicates the genotype.

The phrase “similarity with the reference level” as used herein means that the level of FGL2 is similar to the standard plasma level of FGL2 for a particular genotype, for example genotype 1 or genotype 2/3. HCV has 6 defined genotypes with subgenotypes within many of the defined genotypes. The genotypes are numbered 1-6. At present poor responders are characterized by genotype 1 and 4 whereas genotype 2 and 3 are good responders to antiviral therapy.

It will be appreciated that a very wide variety of assay formats can usefully be adopted for the purpose of detecting the level of polypeptide targets, and any of these formats can be used to detect FGL2. In embodiments, the FGL2 is detected by an immunoassay. In one embodiment, the immunoassay is an enzyme immunoassay, such as a sandwich EIA or enzyme-linked immunosorbant assay (ELISA). In its simplest form, the assay can be performed using the Western format, in which sample is dried onto a suitable substrate such as nitrocellulose, and the dried sample is then probed using a FGL2 binding agent that is either labelled directly or is then reacted with a secondary antibody comprising a detectable label and having binding affinity for the FGL2 binding agent. Washing is introduced at appropriate stages to remove background and unbound reagents. In the alternative, the assay can be performed using a capture agent bound to a solid phase, such as a FGL2 antibody bound to a microtitre well or conjugated to a bead such as a latex or other bead including magnetic beads or fluorescent beads. After mixing the sample and the capture agent, the bound complex is separated from the background and reacted with a detector agent that binds FGL2 at a site different from the capture agent. After isolating or washing the ternary complex, the presence of FGL2 is revealed by the presence of a label associated with the detector agent. If the label is not present on the detector agent, its presence can be established using a secondary antibody that binds the detector reagent and incorporates or is able to generate an appropriate detectable label.

Accordingly, in an embodiment, the level of FGL2 in the plasma sample is determined using an immunoassay. In one embodiment, the immunoassay is an enzyme-linked immunosorbant assay (ELISA). In another embodiment, the ELISA is a sandwich type ELISA.

In another embodiment, the level of FGL2 is determined with an antibody that binds to FGL2. In another embodiment, the antibody is a polyclonal, monoclonal, recombinant or chimeric.

Antibodies that bind FGL2 are described in the literature, and are available commercially. These commercially available antibodies include monoclonal mouse anti-murine FGL2 and monoclonal mouse anti-human FGL2 from Bcroft Inc. Also available are polyclonal anti-mouse FGL2 and polyclonal rabbit anti human FGL2 from Sigma Genosys Tx.

It will be appreciated that antibodies useful in the present assay to bind FGL2 can, in the alternative, be produced de novo by conventional methods, for example, using, as antigen an isolated form of FGL2 or any immunogenic fragment thereof useful to raise antibodies, or a fusion polypeptide comprising a fragment and a carrier that enhances the immune response to the antigen, such as KLH or an Fc fusion. For vaccination, the agent can further be formulated with any adjuvant, such as Freund's, suitable for raising antibody in the selected host. The antibody production host can be any suitable mammal, such as a mouse, rat, rabbit, sheep or goat. Following immunization schedules well established in the art, the desired polyclonal antibody can be extracted from blood using the FGL2 as affinity ligand. To form monoclonal antibodies, splenocytes from immunized animals can then be fused with a selected immortalized partner, and antibody-producing cells can be identified by selection using the FGL2 or fragment thereof as an affinity ligand.

More specifically, antibodies to FGL2 may also be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the present disclosure, antibodies are understood to include monoclonal antibodies, polyclonal antibodies, antibody fragments (e.g., Fab, and F(ab′)₂) and recombinantly produced binding partners.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)); the EBV-hybridoma technique to produce human monoclonal antibodies (Cole et al. Monoclonal Antibodies in Cancer Therapy (1985) Allen R. Bliss, Inc., pages 77-96); and screening of combinatorial antibody libraries (Huse et al., Science 246, 1275 (1989)). Hybridoma cells can be screened immunochemically for production of antibodies specifically reactive with the FGL2 protein and the monoclonal antibodies can be isolated. Therefore, the disclosure also contemplates hybridoma cells secreting monoclonal antibodies with specificity for FGL2.

The term “antibody” as used herein is intended to include fragments thereof which also specifically react with FGL2 or a peptide thereof. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-human animal variable region and a human constant region are also contemplated within the scope of the disclosure. Chimeric antibody molecules can include, for example, the antigen binding domain from an antibody of a mouse, rat, or other species, with human constant regions. Conventional methods may be used to make chimeric antibodies containing the immunoglobulin variable region which recognizes a FGL2 protein (See, for example, Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with the FGL2 as described herein can be further humanized by producing human constant region chimeras, in which parts of the variable regions, particularly the conserved framework regions of the antigen-binding domain, are of human origin and only the hypervariable regions are of non human origin. Such immunoglobulin molecules may be made by techniques known in the art (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982); and PCT Publication WO 92/06193 or EP 0239400). Humanized antibodies can also be commercially produced (Scotgen Limited, 2 Holly Road, Twickenham, Middlesex, Great Britain.)

Specific antibodies, or antibody fragments reactive against FGL2 may also be generated by screening expression libraries encoding immunoglobulin genes, or portions thereof, expressed in bacteria with peptides produced from nucleic acid molecules of the present disclosure. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)).

As an alternative to using antibodies that target FGL2, it will be appreciated that any agent having affinity and binding selectivity for FGL2 is useful to assay FGL2.

The present disclosure also contemplates the use of “peptide mimetics” for detecting FGL2. Peptide mimetics are structures which serve as substitutes for peptides in interactions between molecules (see Morgan and Gainor. (1989), Ann. Reports Med. Chem. 24:243-252 for a review). Peptide mimetics include synthetic structures which may or may not contain amino acids and/or peptide bonds but retain the structural and functional features of binding agents specific for polypeptide products of the biomarkers described in the present disclosure. Peptide mimetics also include peptoids, oligopeptoids (Simon, R. J. et al. Peptoids: a modular approach to drug discovery. Proc. Natl. Acad. Sci. U.S.A (1992) 89, 9367-9371).

The agent is in another embodiment an aptamer. Aptamers can be identified from a library such as a 25 mer library of 4²⁵ random sequences of DNA molecules using the SELEX approach (Systematic Evolution of Ligands by Exponential enrichment).

The term “aptamer” as used herein means a short oligonucleotide that can bind to an antigen eg FGL2. The aforementioned oligonucleotide can be at least 75, 60, 50, 40, 30, 25, 20, 15 or 10 base pairs in length. The term “oligonucleotide” includes DNA and RNA, and can be double stranded or single stranded. In one embodiment, the oligonucleotide is DNA. In a further embodiment, the oligonucleotide is single stranded DNA. The term includes any oligomers or polymers of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides that contain two or more chemically distinct regions. For example, chimeric oligonucleotides may contain at least one region of modified nucleotides that confer beneficial properties (e.g. increased nuclease resistance, increased uptake into cells), or two or more oligonucleotides may be joined to form a chimeric oligonucleotide.

The aptamers of the present disclosure may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil, and 5-trifluoro cytosine.

Aptamers may contain modified phosphorous, oxygen heteroatoms in the phosphate backbone, short chain alkyl or cycloalkyl intersugar linkages or short chain heteroatomic or heterocyclic intersugar linkages. For example, the aptamers may contain phosphorothioates, phosphotriesters, methyl phosphonates, and phosphorodithioates. In an embodiment of the disclosure there are phosphorothioate bonds links between the four to six 3′-terminus bases. In another embodiment phosphorothioate bonds link all the nucleotides.

Aptamers may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an aptamer. Aptamers may also have sugar mimetics.

The aptamers may be constructed using chemical synthesis and enzymatic ligation reactions using procedures known in the art. The aptamers of the disclosure or a fragment thereof, may be chemically synthesized using naturally occurring nucleotides or variously modified nucleotides designed to increase the biological stability of the molecules or to increase the physical stability of the protein-DNA interaction (e.g. phosphorothioate derivatives and acridine substituted nucleotides). The aptamer oligonucleotide sequences may be produced biologically using an expression vector introduced into cells in the form of a recombinant plasmid, phagemid or attenuated virus in which aptamer sequences are produced under the control of a high efficiency regulatory region, the activity of which may be determined by the cell type into which the vector is introduced.

Requisite binding activity is optionally determined by identifying whether binding occurs between the aptamer and FGL2 by “Electrophoretic Mobility Shift Assays (EMSA).” In one embodiment, a useful oligonucleotide is identified when the oligonucleotide complexes with FGL2 and causes upward shift in the oligonucleotide electrophoretic mobility in a DNA retardation gel, such as a 6% polyacrylamide pre-cast DNA retardation gel. Threshold values for a selected aptamer would have its binding capacity from low picomolar affinity to and including 1 microMolar. A person skilled in the art will appreciate that other methods can be used to identify useful variants including flow cytometry, two-photon confocal microscopy, and BIAcore.

It will be appreciated that a variety of labels are suitable for revealing the presence of FGL2 binding agent, and thereby reporting the presence of bound FGL2. Such labels include colloidal gold, which is useful particularly when nitrocellulose strip-based assays are used, as well as radioisotopes, fluorescent markers, luminescent markers, cytochromes, enzymes that catalyze chromogenic substrates, and the like. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as biotin, alkaline phosphatase, beta-galactosidase or horseradish peroxidase; an imaging agent; or a metal ion.

In yet a further aspect, there is provided a method of treating or controlling progression of a viral hepatitis associated with elevated FGL2 levels in plasma, comprising the steps of:

a) identifying a subject having elevated FGL2 as determined by the methods described herein, and

b) treating the subject with an agent that inhibits FGL2 expression or activity.

In one embodiment, the agent is an antibody that inhibits FGL2 or an siRNA molecule against fgl2. Antibodies useful for inhibition include blocking antibodies and can be obtained using methods described herein. The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

The present inventors have developed a reproducible, sensitive and specific enzyme linked immunoassay (ELISA) allowing the measurement of plasma levels of FGL2 in both an experimental murine model of viral hepatitis, mouse hepatitis virus strain 3 (MHV-3) and in patients with chronic hepatitis C virus infection (HCV). Accordingly, the disclosure also provides an assay useful for detecting plasma FGL2 from a subject. In aspects of the disclosure, this assay is exploited for the diagnosis and prognosis of viral hepatitis. Development of a non-invasive, sensitive and specific ELISA assay allows serial monitoring of plasma FGL2 as effector of Treg cells following viral hepatitis and provides a prognosis tool for prediction of disease progression.

Accordingly, in one embodiment, there is provided an assay comprising the steps of:

a) providing a plasma sample from a subject;

b) reacting the sample with an antibody that binds FGL2;

c) detecting bound FGL2; and

d) comparing the level of FGL2 in the sample with the level of FGL2 in a control subject. In one embodiment, the assay is useful in the diagnosis of a viral hepatitis wherein a subject having said viral hepatitis is indicated by a greater level of FGL2 in the sample relative to the level of FGL2 in the control subject. In an embodiment, the plasma sample is first diluted in BSA, such as a dilution of 1/10 in 2.5% BSA.

In an embodiment, the assay is an enzyme-linked immunosorbant assay (ELISA), optionally a sandwich-type ELISA. The ELISA may comprise a capture antibody and a detector antibody. Said antibodies may be polyclonal or monoclonal antibodies.

In another aspect, the present disclosure provides the assays described herein for use the methods of the disclosure.

The assay can be provided in kit form, comprising one or more different and separately packaged agents, including agents that bind to FGL2, together with instructions for the use thereof in performing the assay of the present disclosure. Optionally, the kit may further comprise a quantity of the FGL2, and/or a portion thereof in isolated form for use as a control or calibrator or standard in the assay. The kit may further comprise additional reagents including labelled reagents and other reactants that can be detected using instruments commonly available in the hospital or clinical laboratory.

In one embodiment, the kit comprises an antibody that binds FGL2 and instructions for the use thereof in determining the level of FGL2 in a plasma sample. In another embodiment, the kit further comprises a labelled secondary antibody that binds the FGL2 antibody.

The above generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Materials and Methods

### Reagents

Monoclonal mouse anti-murine FGL2, monoclonal mouse anti-human FGL2, polyclonal rabbit anti-mouse FGL2 (mFGL2) and polyclonal rabbit anti human FGL2 were all produced as previously described (Chan et al. 2003; Liu et al. 2008).

### Generation of Recombinant FGL2

Recombinant mouse and human FGL2 were expressed in the mammalian Chinese hamster ovary (CHO) cell system (Liu et al., 2008). Recombinant FGL2 was purified using a protein A resin column (PALL, Quebec, Canada). Up to 1 mg of recombinant protein was purified from 1 L of cell culture supernatant. Homogeneity of purified FGL2 was evaluated by SDS-PAGE and confirmed by Western blot probed with anti-FGL2 Ab as previously described (Chan et al. 2003; Liu et al. 2008).

### Mice

Female BALB/cJ and A/J mice aged 6 to 8 weeks (Jackson Laboratories) were maintained in micro isolator cages and housed in the animal colony at the Toronto General Hospital, University of Toronto, and fed standard lab chow diet and water ad libitum. The Animal Welfare Committee approved all protocols.

### Viral Infection

MHV-3 was obtained from the American Type Culture Collection, Manassas, Va. It was first plaque-purified and then expanded in murine 17CL1 cells to a concentration of 1×10⁷/ml. Virus-containing supernatants were collected and subsequently stored at −80° C. until use. Mice were infected with 100 PFU by the intraperitoneal (ip) route. Mice received an ip injection of 100 PFU MHV-3 and were monitored daily for symptoms of disease, including ruffled fur, tremors, and lack of activity.

### Mouse and Human Blood Preparation

Mice were sacrificed on days 0, 1, 2, 3 and 8 (only A/J mice) post-MHV-3 infection. 350 μl of blood was collected via cardiac puncture into Eppendorf tubes containing 10 μl of Heparin 1000 u/ml and iced immediately. This was centrifuged at 500×g for 10 minutes. The plasma was collected and frozen at −80° C. for future analysis.

For human sample preparation, blood samples were collected from healthy volunteers and patients with biopsy proven chronic HCV infection and alcohol induced cirrhosis and were centrifuged at 1500×g for 10 minutes at 21° C. in heparinized tubes. Plasma was subsequently collected and frozen at −80° C. Human ethical committee from Toronto General Hospital approved the study.

### Sandwich ELISA for FGL2

The standard sandwich ELISA method was used for measurement of plasma levels of FGL2 (Crowther, 1995). Briefly, a 96-well microplate (Costar EIA/RIA 96-well flat-bottomed plate, Corning Inc. Corning, N.Y.) was coated at 4° C. overnight with 50 ul of 1 ug/ml monoclonal mouse anti-murine or anti-human FGL2 antibody respectively. After washing in phosphate-buffer saline (PBS) containing 0.05% Tween-20 (washing buffer) three times, the plate was incubated with 230 μl/well of Superblock solution (Pierce Biotechnology, Inc. Rockford, Ill.) at room temperature for one hour. 50 ul of recombinant mouse or human FGL2 (standards), mouse plasma or patient plasma (diluted 1/10 with 2.5% BSA) was added to each well in 4-6 replicates and incubated for 1 hour followed by reaction for 1 hr with 50 ul/well of polyclonal rabbit anti-mouse or anti-human FGL2 antibody (1 ug/ml in 1% BSA in PBS). The plate was washed in washing buffer five times. The goat anti-rabbit IgG-h+1 HRP (horseradish peroxidase) conjugated cross-absorbed secondary antibody (Bethyl laboratories Inc, Montgomery Tex.) was diluted to 1 ug/ml with 1% BSA in PBS and 50 ul was added to each well. The development was performed using 100 μl/well of tetramethyl benzidine (Sigma Aldrich, Oakville, ON), at room temperature for 5 min and was stopped by adding 100 μl of 2M H₂SO₄. Absorbance was measured as OD 450 nm using Appliskan multimode microplate reader (ThermoFisher Scientific Inc, Waltham Mass.).

### Immunostaining for FGL2

Formalin-fixed, paraffin-embedded tissue from explanted liver of chronic hepatitis C patients were sectioned and heat-retrieved in 20 mM Tris/0.65 mM EDTA/0.0005% Tween at pH 9.0. Endogenous peroxidase activities were blocked using 3% aqueous hydrogen peroxide and stained overnight at room temperature with a mouse monoclonal antibody against FGL2 (homemade, clone 9D8) at 1:1000 dilution in a moist chamber. The staining was finished by using the UltraVision LPValue HRP Polymer kit (Lab Vision) according to manufacturer's instructions. FGL2 staining was detected by horse radish peroxidase (HRP) staining whereas FoxP3 was detected by staining with tetrazolium blue (Marsden et al. 2003; Ning et al. 1999).

### Statistical Analysis

Results are reported as mean and standard error of mean (SEM) unless otherwise specified. One-way or two-way ANOVA followed by the Bonferroni test for post-hoc analysis were used for group comparison. Differences with p<0.05 were considered significant.

### Results

### 1—Sensitivity, Specificity and Reproducibility of the ELISA

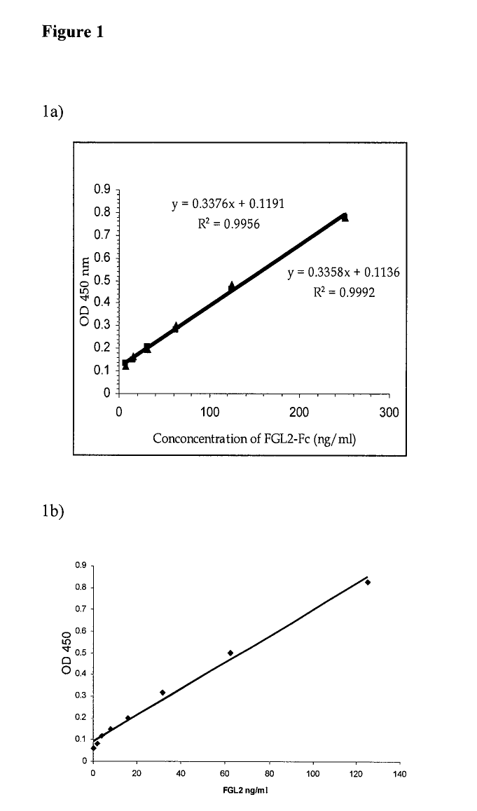

In order to assess the sensitivity of the assay, a serial dilution of known concentrations of recombinant FGL2 were used to establish a standard curve for FGL2. The standard dose-response curve of both mouse and human ELISA system was linear when plotted on a log/log scale (R²=0.99). The detectable range was between 7.8 to 500 ng/ml for mouse and 0.3 to 125 ng/mL for humans respectively (FIGS. 1a and 1b). The specificity of the assay was determined by measurement of plasma levels of FGL2 obtained from mice with targeted deletion of fgl2 (fgl2^{-/-}). No detection was observed by the ELISA indicating that the assay specificity for mouse FGL2 with no cross-reactivity with other proteins.

To assess the reproducibility of the serial measurement of the standard for the mouse assay and the human assay were performed. The mean optical density (OD) values with standard deviation, and coefficients of variation within the same assay and between the assays are provided in Table 1 a &b.

### 2—Plasma Levels of FGL2 in Murine Hepatitis Virus Strain 3 (MHV-3) Infection

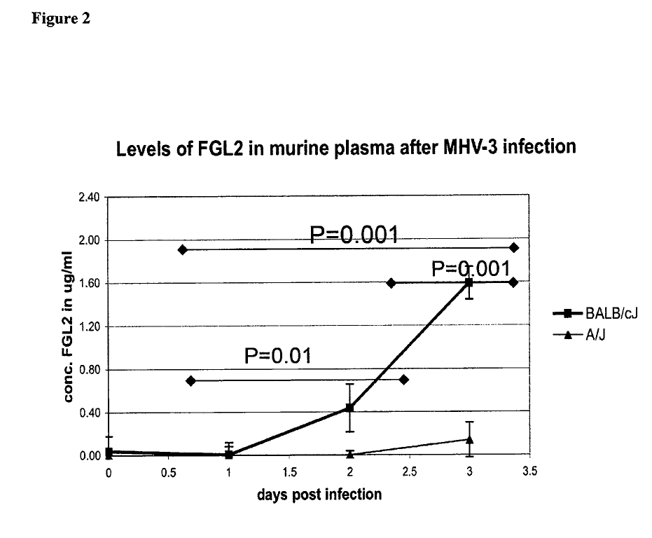

The present inventors have extensively studied the role of FGL2 in a model of fulminant hepatitis caused by murine hepatitis virus strain 3 (MHV-3) (Shalev et al. 2009). Susceptible strains of mice (BALB/cJ) develop a fatal hepatitis within 3-5 days post infection that is characterized by intravascular thrombosis and hepatocellular necrosis; resistant strains of mice (A/J) survive and clear the virus within 10-14 days of infection. Several lines of evidence suggest that induction of FGL2 contributes to the lethality of MHV-3 induced hepatitis. First, only in susceptible animals was there an induction of FGL2 by MHV-3 (Shalev et al. 2009). Second, treatment of mice with a monoclonal antibody to FGL2 prevents thrombosis, hepatic necrosis and lethality of MHV-3 infection (Shalev et al. 2009). Using the presently described ELISA, plasma samples were analyzed from both MHV-3 uninfected and infected susceptible and resistant mice daily from the time of infection to day 3 post infection (FIG. 2). At baseline (prior to MHV-3 infection) plasma levels of FGL2 were higher in mice susceptible to MHV-3 (BALB/cJ) (124±36 ng/ml) in comparison to resistant mice (A/J) (79±17 ng/ml) (P<0.01). Following infection with MHV-3, plasma levels of FGL2 correlated with disease progression. Mice which were susceptible to the virus (BALB/cJ) and developed fulminant hepatitis expressed significantly higher levels of FGL2 over time post infection with a maximal level of 1589±75 ng/ml seen on day 2 post infection just prior to death whereas mice resistant to the virus (A/J) had no significant increase in levels of FGL2 similar to basal levels (<0.09 μg/ml).

### 3—Plasma Levels of FGL2 in Healthy Human Controls

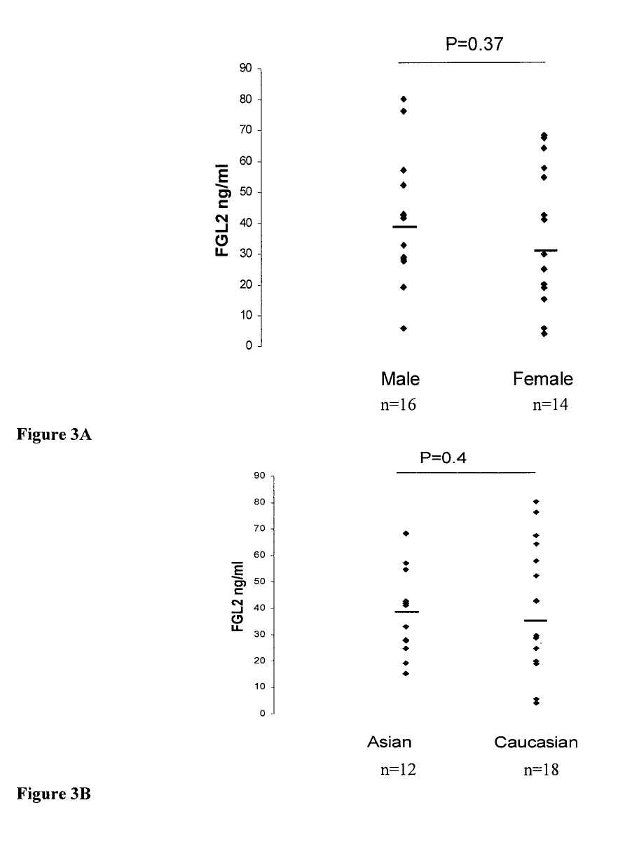

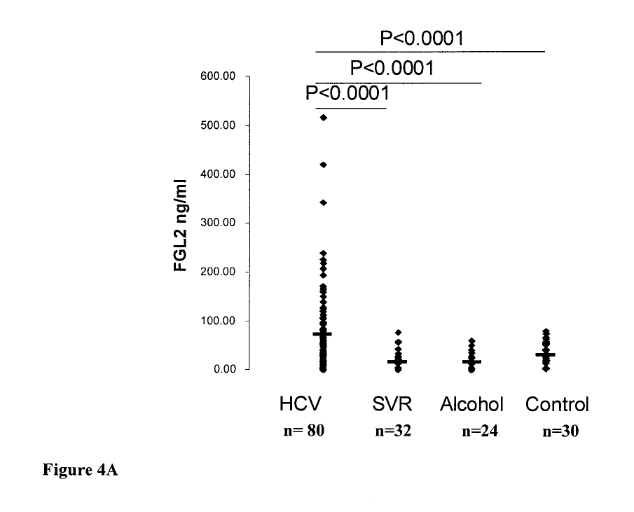

To establish the baseline level of plasma FGL2 in human 10 ml of plasma was collected from 36 healthy controls. The baseline characteristics of these human controls are presented in Table 2. Plasma levels of FGL2 were measured by the human ELISA. Mean plasma levels of FGL2 in healthy controls were 36.41±21.9 ng/mL. No significant difference in mean plasma levels of FGL2 was observed between Males vs. Females (38.8±23.4 ng/mL vs. 34.2±22.0 ng/mL, respectively) or Caucasians vs. Asians (37.9±22.9 ng/mL vs. 35.3±22.4 ng/mL, respectively) (FIGS. 3A and 3B).

4—Plasma Levels of FGL2 in Patients with Chronic HCV Infection

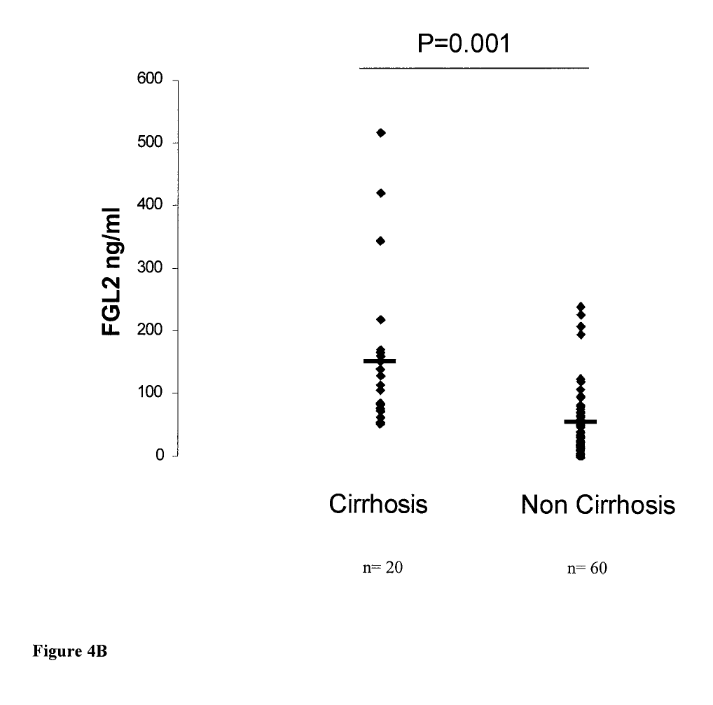

To further assess the hypothesis that plasma levels of FGL2, a known immunoregulatory effector of Treg cells, is increased in patients with chronic HCV infection, the plasma levels of FGL2 were measured in 80 patients with biopsy proven chronic active HCV or patients with alcohol induced cirrhosis. Baseline characteristics of these patients are presented in Table 2. Mean plasma levels of FGL2 were significantly higher in patients with chronic HCV infection (84.3±89.1 ng/mL, n=80) compared to healthy controls (36.41±21.9 ng/mL, n=30, p<0.001) or to patients with alcoholic cirrhosis (18.8±17.4 ng/mL, n=24, p<0.001), and HCV patients who had received antiviral therapy and reached a sustained virologic response (SVR) (16.6±19.7 ng/ml, n=32, p<0.001), (FIG. 4A). Among HCV patients, mean plasma levels of FGL2 were significantly higher in patients with cirrhosis (164.1±121.8 ng/mL, n=20) compared to non cirrhotics (57.7±52.8 ng/mL, n=60, p=0.001) (FIG. 4B).

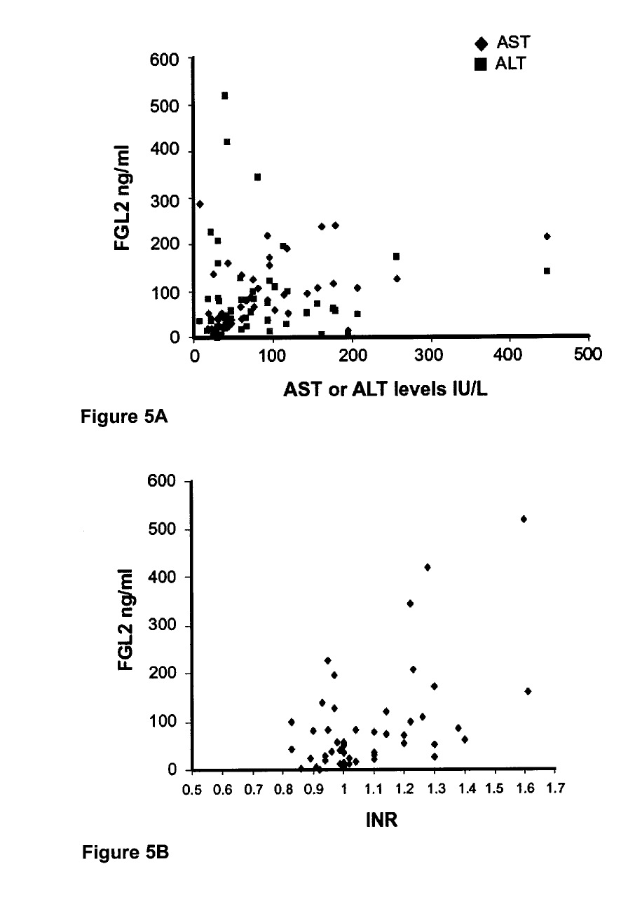

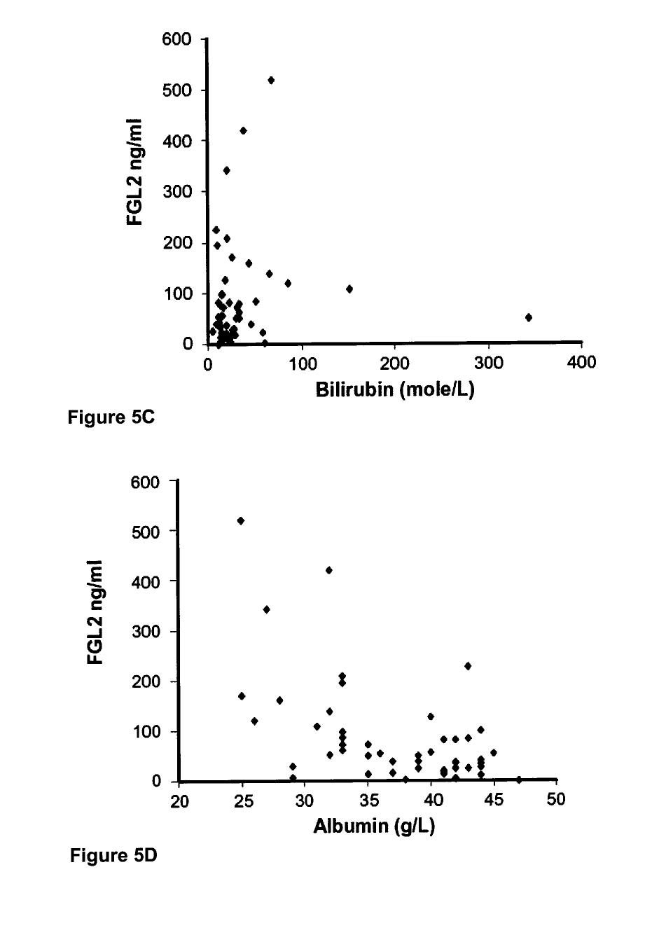

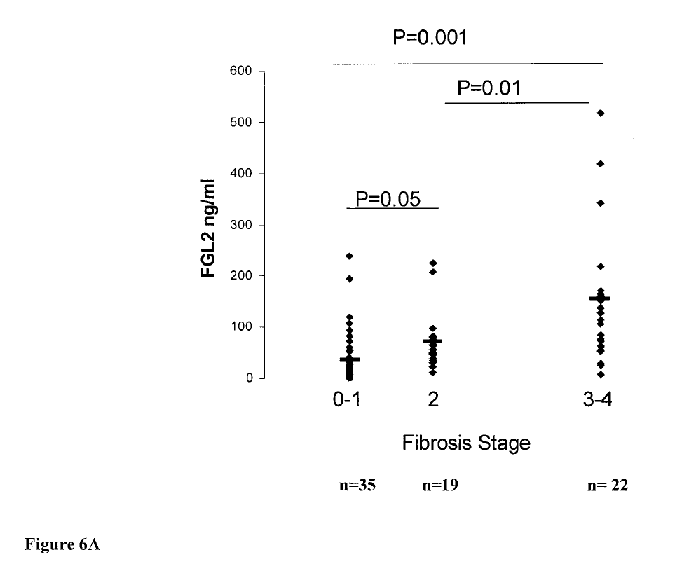

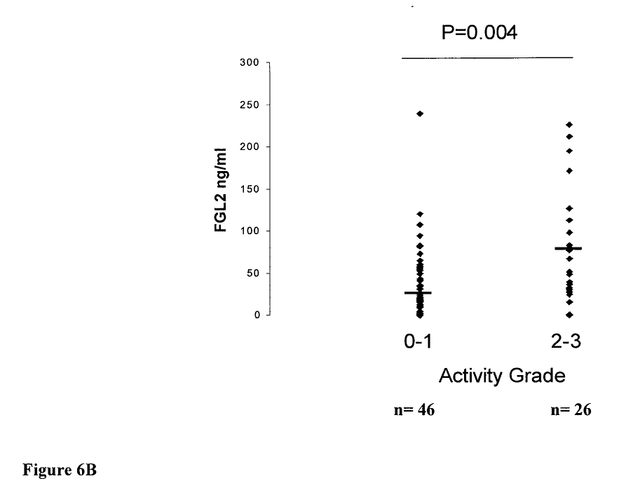

Levels of FGL2 correlated positively and significantly with serum levels of aspartate transaminase (AST) (Rs=0.50, p=0.0001) (FIG. 5A), prothrombin time (INR) (Rs=0.46, p=0.0001) (FIG. 5B), and bilirubin (Rs=0.41 p=0.001) (FIG. 5C) but only weakly with levels of alanine transaminase (ALT) (Rs=0.21, p=NS) (FIG. 5A). There was a negative correlation of levels of FGL2 with serum albumin (Rs=−0.32, p=NS) (FIG. 5D). Fibrosis stage is a well known marker for disease severity and indication for anti-viral treatment. Liver biopsies performed within 12 months of collecting blood for FGL2 determination were available for assessment of fibrosis stage (Metavir) in 76 of the 80 patients with HCV infection. None of the patients were treated for HCV with antiviral therapy during the course of this study. FGL2 plasma levels were significantly higher in patients with advanced fibrosis (stage 3-4, n=22) compared to the patients with mild fibrosis (stage≦1, n=35) (148.3+143.0 vs. 44.4+52.4 ng/ml, p=0.001) and moderate fibrosis (stage 2, n=19) (148.3+143.0 vs. 72.9+65.9 ng/ml, p=0.01) (FIG. 6A). Furthermore, HCV patients with stage 2 fibrosis had significantly higher levels of FGL2 compared to patients with stage≦1 (72.9+65.9 vs. 44.4+52.4 ng/ml, p=0.05). Similarly, analysis of plasma levels of FGL2 according to the activity grade (METAVIR) (FIG. 6B) showed a significant difference between patients with higher inflammation as assessed by activity grade (grade≧2, n=26), compared to those with lower grade of inflammation (grade<2, n=46) (74.4+69.8 vs. 36.8+37.1 ng/ml, p=0.01).

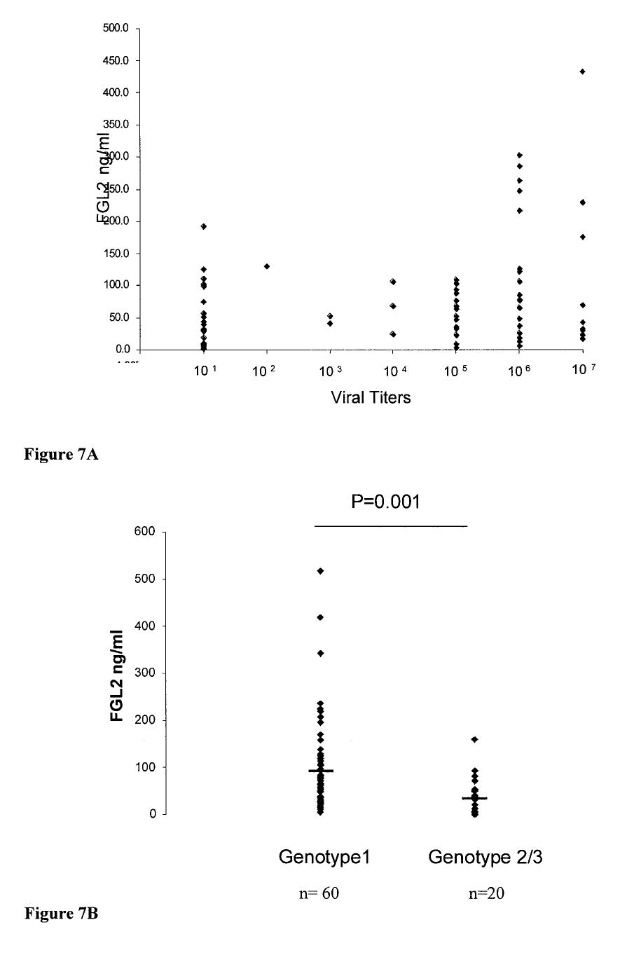

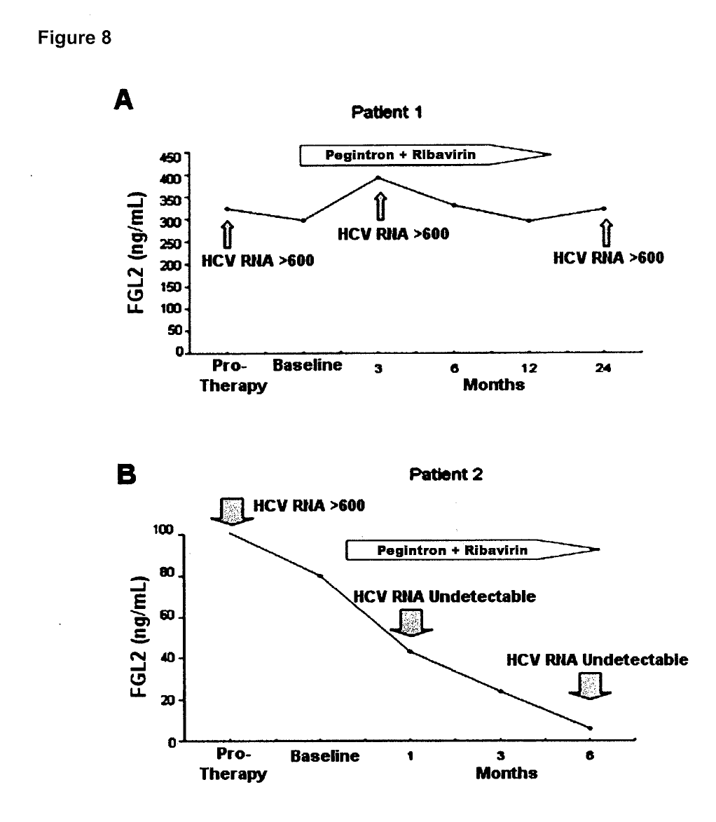

Plasma levels of FGL2 did not correlate significantly with viral titers (FIG. 7A). Analysis of the plasma levels of FGL2 according to the HCV genotype showed that genotype 1 patients, known to have lower response rates to anti-viral therapy, had significantly higher levels of plasma FGL2 compared to genotype 2/3 patients (98.0±100.3 vs. 41.5±38.6 ng/ml, p=0.0008) (FIG. 7). Furthermore, the mean plasma levels of FGL2 in a subset of HCV patients (n=32), who had cleared the virus following treatment with pegylated interferon (alpha 2b 1.5 μg/kg body weight. s.c. once weekly or pegylated interferon alpha 2b 180 μg s.c. once weekly) and ribavirin (800 mg [body weight<75 kg] or 1000 mg [body weight≧75 kg] p.o. in two divided doses) and developed a sustained virological response (SVR) for more than 6 months post therapy, were significantly lower compared to patients with active disease (16.6+19.7 vs. 84.3+89.1 ng/ml, n=80, p<0.001) (FIG. 4A). In one patient with chronic HCV genotype 1 infection who failed to clear HCV following treatment with pegylated interferon and ribavirin, levels of FGL2 were markedly elevated prior to treatment (320.4 ng/mL) and remained elevated throughout the course of anti viral therapy. In contrast in another patient with chronic HCV genotype 2 infection who responded to anti viral therapy, levels of FGL2 at baseline were only minimally elevated (80.9 ng/ml) and within 1 month of therapy they returned to levels seen in normal healthy controls and was undetectable at the end of treatment (FIG. 8).

5—FGL2 Expression in the Liver of Patients with Chronic HCV Infection

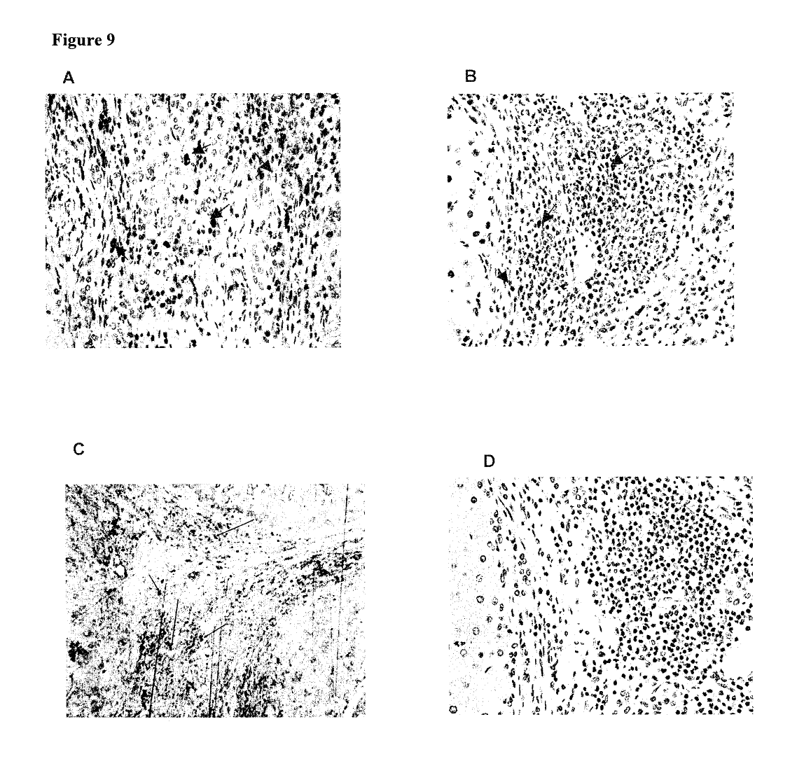

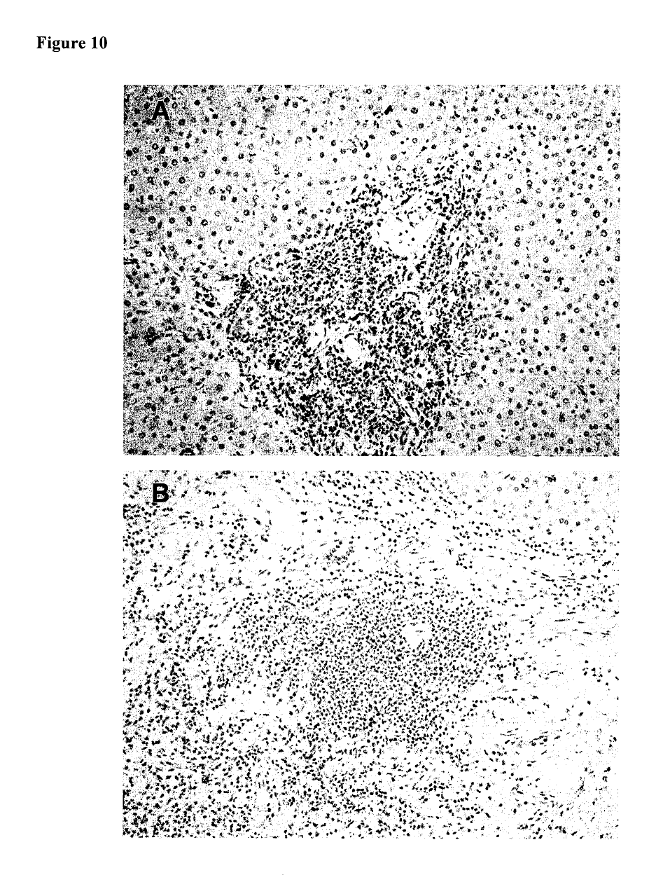

To evaluate the expression of FGL2 in the liver, immunostaining of FGL2 protein and the transcription factor FoxP3 was performed in explanted livers of HCV patients who had undergone liver transplantation. Typical features of HCV histopathology were observed including a heavy infiltration of lymphocytes and macrophages in both the portal and periportal areas (FIG. 9A). Both FGL2 and Fox P3+ immunostaining were observed within infiltrating cells (FIGS. 9B and C). There were significantly more FoxP3⁺ cells than FGL2⁺ cells. FoxP3 was localized to the nucleus whereas FGL2 was seen predominantly in the cytoplasm and at the cell surface. FoxP3/FgL2 co-staining showed that only a small but significant proportion of lymphocytes stained for both FGL2 and FoxP3 and that the majority of FoxP3⁺ cells were FGL2 negative supporting recent reports that not all FoxP3⁺ cells are Tregs (FIG. 9D) (Gonzalez-Quintela et al. 2000). Two patients were studied in more detail. One patient, a 49 year old male had contracted HCV, genotype 1 approximately 15 years earlier. He had histologic evidence of severe chronic HCV disease with dense periportal and portal lymphocyte infiltrates and grade 2-3 fibrosis. This patient was found to have numerous FGL2 positive infiltrating lymphocytes and was treated with pegylated interferon and ribavirin for 48 weeks but failed to clear HCV (FIG. 10A). In contrast, a second patient (53 year old male) who also had evidence of chronic HCV genotype 1 disease histologically had no detectable FGL2 positive cells within the biopsy. Following treatment for 48 weeks with pegylated interferon and ribavirin this patient cleared the virus (FIG. 10B).

### Discussion

In the present study, the development of a reproducible, sensitive and specific enzyme linked immunoassay (ELISA) was described enabling the measurement of plasma levels of FGL2, a novel effector of Tregs in both humans and in mice. Using the FGL2 ELISA, plasma levels of FGL2 were measured in an experimental model of mouse viral hepatitis and in healthy controls as well as in patients with chronic hepatitis C virus infection. The murine and human FGL2 ELISA assays were capable of measurement of plasma levels of FGL2 over a wide range and with excellent reproducibility. Furthermore, in mice susceptible to MHV-3 infection, plasma levels of FGL2 were predictive of susceptibility and severity of disease. The increased levels of FGL2 seen in susceptible BALB/cJ mice pre infection correlated with their known TH2 phenotype, inability to mount a protective anti viral TH1 response and increased numbers of CD4+CD25+FoxP3+ Treg which have been shown to be critical to the pathogenesis of MHV-3 induced liver disease (Shalev et al. 2009). It was further demonstrated that patients with HCV infection, had significantly higher plasma levels of FGL2 than healthy controls and patients with end stage alcoholic cirrhosis. Patients with genotype 1, known to have more aggressive disease and poorer response to treatment with anti viral therapy had significantly higher levels of FGL2 than patients with genotype 2 and 3. Furthermore, in HCV patients who cleared the virus following antiviral therapy, levels of FGL2 returned to levels seen in normal healthy controls. Collectively, these results suggest that measurement of FGL2 may not only be useful as a diagnostic test but more importantly may identify patients who will benefit from antiviral therapy. A randomized controlled study is now underway to examine this (www.clinicaltrials.gov).

In the present study, monitoring plasma levels of FGL2 were shown to be of use to predict the activity and severity of both MHV-3 induced FH and chronic HCV in humans. Thus this non-invasive, sensitive and specific ELISA assay appears to provide a prognostic tool for prediction of disease progression in contrast to the present series of tests available including measurement of liver biochemistry and viral titres.

The FGL2 ELISA assay is highly sensitive and specific for measurement of human and mice FGL2. The present inventors did not observed any cross reactivity with other plasma proteins, and no reading was obtained using serum from mice with targeted depletion of Fgl2. Serial measurement of FGL2 levels in mice before and following infection with mouse hepatitis virus strain 3 (MHV-3) showed a significant difference between animals which are susceptible and resistant to the viral infection. The difference in FGL2 level pre and post infection correlated well with the degree of disease progression since in susceptible mice plasma FGL2 levels raised proportionally to the severity of disease with a highest level prior to the animal death. In contrast, in resistant mice plasma level of FGL2 only increased minimally compared to the baseline level. Furthermore, the difference in plasma levels of FGL2 between the two strains of mice corresponded well to the Th2/Th1 immune responses phenotypes exhibited by these mice. High plasma levels of FGL2 in susceptible BALB/cJ mice fits with demonstrated effect of FGL2 to promotes with Th2 immune response (Chan et al. 2003) which is the reported phenotype exhibit by BALB/cJ mice following MHV-3 infection. In contrast, in resistant A/J mice which mount predominantly Th1 immune responses, plasma level of FGL2 did not increase significantly following viral infection. Collectively, these results indicate that plasma level of FGL2 could be a marker for predication of disease progression and severity following viral infection.

One interesting observation in the present study is the wide range of plasma FGL2 level (10.2-550.6 ng/ml) in patients with HCV infection. One explanation for this variability might be that patients with lower level of FGL2 are those which have a higher probability to respond successfully to antiviral treatment in contrast to those with higher levels. Previous results demonstrating that FGL2 levels correlated with the numbers of Treg cells observed in both resistant and susceptible strains of mice to MHV-3 (Shalev et al. 2009) as well as development of Th2 response in susceptible mouse to viral infection further support this hypothesis. These results suggest that the levels of FGL2 expressed by Treg cells may determine, at least in part, the type of immune response that will be generated and ability to respond to antiviral therapy. A controlled clinical trial is now underway to confirm this hypothesis.

Similarly, in patients with genotype 1, plasma levels of FGL2 were significantly higher compared to the patients with genotypes 2/3. It is well established that HCV patients with genotype 2/3 have a higher rate of response to antiviral therapy (70-80% sustained virological response) compared to the patients with genotype 1 (50% SVR). These results again support the hypothesis that patients with lower levels of FGL2 indicative of Treg activity have a higher chance of viral clearance following antiviral therapy.

Liver biopsies from patients with chronic HCV showed severe disease as indicated by marked portal and periportal cellular infiltrates and liver fibrosis. In the livers of some patients, there were increased numbers of FoxP3+ T cells which stained for FGL2 both within the cytoplasm of cells and at the cell surface. As opposed to findings in mice, in which all FoxP3+ cells were FGL2 positive, many FoxP3+ cells were FGL2 negative (Shalev et al. 2009). This is consistent with the recent reports by Allan et al. who has demonstrated that in humans FoxP3 is not a specific marker for Treg and can be found in T effector cells (Allan et al. 2008). Of interest, some patients had few if any FoxP3+ cells within the liver and absence of FGL2 staining and this finding corresponded to a better response to anti viral therapy. These results support our hypothesis that patients with lower levels of FGL2 indicative of low numbers of Treg have a higher chance of viral clearance following antiviral therapy.

In summary, a sensitive, specific and accurate ELISA has been established which allows measurement of plasma FGL2 both in mice and in humans. The results suggest that monitoring plasma level of FGL2 in patients with chronic HCV infection might be a mechanism for predicting disease severity as well as response to antiviral therapy. A prospective clinical trial has been initiated in patients with chronic HCV infection to further assess the utility of this assay in predicting both severity of disease and response to antiviral therapy. Interference with FGL2 and Treg activity may allow successful anti viral therapy.

### Example 2

### Plasma Levels of FGL2 in HBV Patients

Twenty-three patients with biochemical, histological, and clinical evidence of marked chronic viral hepatitis B and 13 patients with minimal chronic viral hepatitis B were studied for hepatic Fgl2/fibroleukin expression. Patient characteristics are noted in Table 3. Of note, HBsAg, HBeAg, anti-HBcAg and hepatitis B viral DNA load did not correlate with expression of Fgl2/fibroleukin. Although ALT and total bilirubin levels tended to be higher in patients with marked chronic hepatitis versus minimal chronic hepatitis B, there was considerable overlap in patient values. Twenty-one of the 23 patients with marked chronic viral hepatitis B had elevated Fgl2/fibroleukin levels.

Table 3 shows the characteristics of patients with Hepatitis B virus that were used to measure FGL2 plasma levels. An ELISA as described in Example 1 was used to measure the plasma levels of HBV patients and it was shown that patients having severe chronic active hepatitis (CAH) exhibited significantly increased plasma FGL2 (13.6+/−6.8 ng/mL) compared to normal controls (4.8+/−2.6 ng/mL) or minimal CAH (5.2+/−2.1 ng/mL) (P<0.001) (Table 4).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Reproducibility of mouse and human ELISA assay
1a) mouse ELISA

FGL2-Fc (ng/ml)

| | n | Mean | STD | % CV |
|---|---|---|---|---|
| Intra-assay | 5 | 7.81 | 0.54 | 6.9 |
| variation | 5 | 1.95 | 0.09 | 4.6 |
| | 5 | 0.1 | 0.008 | 8 |
| Inter-assay | 4 | 15.6 | 0.85 | 5.5 |
| variation | 4 | 2.5 | 0.23 | 9.2 |
| | 4 | 0.1 | 0.002 | 2 |

1b) human ELISA

FGL2 ng/ml

| | N* | Mean | STD | % CV |
|---|---|---|---|---|
| Intra-assay | 4 | 60.6 | 0.89 | 1.4 |
| variation | 4 | 15.6 | 0.62 | 3.9 |
| | 4 | 3.6 | 0.26 | 7.2 |
| Inter-assay | 4 | 54.3 | 1.65 | 3 |
| variation | 4 | 6.7 | 0.29 | 4.3 |
| | 4 | 1.8 | 0.13 | 7.2 |

TABLE 2
Baseline characteristics of healthy volunteers
and patients with chronic HCV infection

| | | | | Alcoholic |
|---|---|---|---|---|
| | Healthy controls | HCV patients | | patients |
| | N^{A} = 30 | N = 80 | | N = 24 |
| Age mean (range) | 32 (18-56) | 60 | (25-65) | 65 (45-69) |
| Female N (%) | 14 (46) | 14 | (17) | 6 (25) |
| Caucasian N (%) | 18 (60) | 58 | (72.5) | 23 (95) |
| Asian N (%) | 12 (40) | 20 | (25) | 1 (5) |
| African-american | NA | 2 | (2.5) | NA |
| N (%) | | | | |

| | | | |
|---|---|---|---|
| Genotype 1 N | NA | 60 | NA |
| Genotype 2/3 N | NA | 20 | NA |

(^{A}N is number of patients)

TABLE 3
Characteristics of the Patients with Hepatitis B Virus

| | Minimal chronic | Marked chronic |
|---|---|---|
| Characteristic | hepatitis B (n = 13) | hepatitis B (n = 23) |
| Age (year) | | |
| Mean | 43.8 | 36.0 |
| SD | 5.6 | 7.8 |
| Male sex (%) | 85 | 87 |
| HBsAg (%) | 100 | 100 |
| HBeAg (%) | 46 | 30 |
| Anti-HBcAg (%) | 85 | 91 |
| HBV DNA | 54 | 39 |
| Serum ALT (IU/L) | | |
| Mean | 300.5 | 586.0 |
| SD | 238.9 | 404.2 |
| Serum bilirubin (umol/L) | | |
| Mean | 78.3 | 452.1 |
| SD | 89.4 | 191.7 |

TABLE 4
FGL2 ELISA for Patients with HBV

| Patient Population | Plasma FGL2 (ng/mL) | P value |
|---|---|---|
| Normal (n = 36) | 4.8 ± 2.6 | |
| Minimal CAH (n = 13) | 5.2 ± 2.1 | NS |
| Severe CAH (n = 23) | 13.6 ± 6.8 | <0.001 |

P compared to normal controls

### REFERENCES

Allan S E, Song-Zhao G X, Abraham T, McMurchy A N, Levings M K. Inducible reprogramming of human T cells into Treg cells by a conditionally active form of FOXP3. Eur J Immunol 2008; 38:3282-3289.
Bach J F, Chatenoud L. Tolerance to islet autoantigens in type 1 diabetes. Annu Rev Immunol 2001; 19:131-161.
Belkaid Y, Rouse B T. Natural regulatory T cells in infectious disease. Nat Immunol 2005; 6:353-360.
Boettler T, Spangenberg H C, Neumann-Haefelin C, Panther E, Urbani S, Ferrari C, Blum H E, et al. T cells with a CD4+CD25+ regulatory phenotype suppress in vitro proliferation of virus-specific CD8+ T cells during chronic hepatitis C virus infection. J Virol 2005; 79:7860-7867.
Cabrera R, Tu Z, Xu Y, Firpi R J, Rosen H R, Liu C, Nelson D R. An immunomodulatory role for CD4(+)CD25(+) regulatory T lymphocytes in hepatitis C virus infection. Hepatology 2004; 40:1062-1071.
Chan C W, Kay L S, Khadaroo R G, Chan M W, Lakatoo S, Young K J, Zhang L, et al. Soluble fibrinogen-like protein 2/fibroleukin exhibits immunosuppressive properties: suppressing T cell proliferation and inhibiting maturation of bone marrow-derived dendritic cells. J Immunol 2003; 170:4036-4044.
Doolittle, R. F. 1983. The structure and evolution of vertebrate fibrinogen. Ann N Y Acad Sci 408:13-27.
Fontenot J D, Rasmussen J P, Gavin M A, Rudensky A Y. A function for interleukin 2 in Foxp3-expressing regulatory T cells. Nat Immunol 2005; 6:1142-1151.
Furuichi Y, Tokuyama H, Ueha S, Kurachi M, Moriyasu F, Kakimi K. Depletion of CD25+CD4+T cells (Tregs) enhances the HBV-specific CD8+ T cell response primed by DNA immunization. World J Gastroenterol 2005; 11:3772-3777.
Gonzalez-Quintela A, Dominguez-Santalla M J, Perez L F, Vidal C, Lojo S, Barrio E. Influence of acute alcohol intake and alcohol withdrawal on circulating levels of IL-6, IL-8, IL-10 and IL-12. Cytokine 2000; 12:1437-1440.
Hancock W W, Szaba F M, Berggren K N, Parent M A, Mullarky I K, Pearl J, Cooper A M, et al. Intact type 1 immunity and immune-associated coagulative responses in mice lacking IFN gamma-inducible fibrinogen-like protein 2. Proc Natl Acad Sci USA 2004; 101:3005-3010.
Herman A E, Freeman G J, Mathis D, Benoist C. CD4+CD25+ T regulatory cells dependent on ICOS promote regulation of effector cells in the prediabetic lesion. J Exp Med 2004; 199:1479-1489.
Levy G A, Leibowitz J L, Edgington T S. Induction of monocyte procoagulant activity by murine hepatitis virus type 3 parallels disease susceptibility in mice. J Exp Med 1981; 154:1150-1163.
Levy G A, Liu M, Ding J, Yuwaraj S, Leibowitz J, Marsden P A, Ning Q, et al. Molecular and functional analysis of the human prothrombinase gene (HFGL2) and its role in viral hepatitis. Am J Pathol 2000; 156:1217-1225.
Liu H, Shalev I, Manuel J, He W, Leung E, Crookshank J, Liu M F, et al. The FGL2-FcgammaRIIB pathway: a novel mechanism leading to immunosuppression. Eur J Immunol 2008; 38:3114-3126.
Marazzi S, Blum S, Hartmann R, Gundersen D, Schreyer M, Argraves S, von Fliedner V, et al. Characterization of human fibroleukin, a fibrinogen-like protein secreted by T lymphocytes. J Immunol 1998; 161:138-147.
Marsden P A, Ning Q, Fung L S, Luo X, Chen Y, Mendicino M, Ghanekar A, et al. The Fgl2/fibroleukin prothrombinase contributes to immunologically mediated thrombosis in experimental and human viral hepatitis. J Clin Invest 2003; 112:58-66.
Miyara M, Sakaguchi S, Natural regulatory T cells: mechanisms of suppression. Trends Mol Med 2007; 13:108-116.
Ning Q, Liu M, Kongkham P, Lai M M, Marsden P A, Tseng J, Pereira B, et al. The nucleocapsid protein of murine hepatitis virus type 3 induces transcription of the novel fgl2 prothrombinase gene. J Biol Chem 1999; 274:9930-9936.
Rushbrook S M, Ward S M, Unitt E, Vowler S L, Lucas M, Klenerman P, Alexander G J. Regulatory T cells suppress in vitro proliferation of virus-specific CD8+ T cells during persistent hepatitis C virus infection. J Virol 2005; 79:7852-7859.
Sakaguchi S, Naturally arising Foxp3-expressing CD25+CD4+ regulatory T cells in immunological tolerance to self and non-self. Nat Immunol 2005; 6:345-352.
Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol 1995; 155:1151-1164.
Shalev I, KM W, K F, Y Z, C C, A M, J Z, et al. The novel CD4+CD25+ Regulatory T Cell Effector Molecule Fibrinogen-like Protein 2 contributes to the Outcome of Murine Fulminant Viral Hepatitis. Hepatology 2009; 49:387-397.
Shalev I, Liu H, Koscik C, Bartczak A, Javadi M, Wong K M, Maknojia A, et al. Targeted deletion of fgl2 leads to impaired regulatory T cell activity and development of autoimmune glomerulonephritis. J Immunol 2008; 180:249-260.
Stoop J N, van der Molen R G, Baan C C, van der Laan L J, Kuipers E J, Kusters J G, Janssen H L. Regulatory T cells contribute to the impaired immune response in patients with chronic hepatitis B virus infection. Hepatology 2005; 41:771-778.
Williams L M, Rudensky A Y. Maintenance of the Foxp3-dependent developmental program in mature regulatory T cells requires continued expression of Foxp3. Nat Immunol 2007; 8:277-284.
Wood K J, Sakaguchi S. Regulatory T cells in transplantation tolerance. Nat Rev Immunol 2003; 3:199-210.
Xu D, Fu J, Jin L, Zhang H, Zhou C, Zou Z, Zhao J M, et al. Circulating and liver resident CD4+CD25+ regulatory T cells actively influence the antiviral immune response and disease progression in patients with hepatitis B. J Immunol 2006; 177:739-747.
Zager R A, Johnson A C, Lund S. ‘Endotoxin tolerance’: TNF-alpha hyper-reactivity and tubular cytoresistance in a renal cholesterol loading state. Kidney Int 2007; 71:496-503.

1. A method for identifying a subject having an elevated FGL2 level in plasma, the method comprising the step of assaying a plasma sample from the subject to determine the level of FGL2, wherein a level above control indicates that the subject has elevated FGL2 levels.

2. The method according to claim 1 for identifying a subject having or at risk for a viral hepatitis associated with elevated FGL2 in plasma, the method comprising the steps of:
a) providing a plasma sample from said subject; and
b) determining a level of FGL2 in said sample,
wherein a FGL2 level above control indicates the subject has or is at risk for said viral hepatitis.

3. The method according to claim 1 for monitoring disease progression in a subject of a viral hepatitis associated with elevated FGL2 in plasma, the method comprising the steps of:
a) at a first time point, determining the level of FGL2 in a plasma sample from the subject; and
b) comparing the level of FGL2 in a plasma sample taken from said subject at a second time point different from said first time point;
wherein a difference in the FGL2 levels at the first time point compared to the second time point indicates modulated progression of the viral hepatitis.

4. The method according to claim 1 for monitoring the response to anti-viral therapy in a subject infected with viral hepatitis, the method comprising the steps of:
a) at a first time point, determining the level of FGL2 in a plasma sample from the subject; and
b) comparing the level of FGL2 in a plasma sample taken from said subject at a second time point different from said first time point;
wherein a decrease in the FGL2 levels at the second time point compared to the first time point indicates response to anti-viral therapy.

5. The method according to claim 1 for predicting the response to anti-viral therapy and clearance of the virus in a subject infected with viral hepatitis, the method comprising the steps:
a) determining the level of FGL2 in a plasma sample from the subject prior to initiation of antiviral therapy;
b) comparing the level in a) to a control;
wherein a FGL2 level above control indicates non response to antiviral therapy.

6. The method according to claim 1 for genotyping a subject infected with viral hepatitis comprising the steps:
a) assaying plasma from the subject to determine a level of FGL2; and
b) comparing to a reference level;
wherein similarity with the reference level indicates the genotype.

7. A method of treating or controlling progression of a viral hepatitis associated with elevated FGL2 levels in plasma, comprising the steps of:
a) identifying a subject having elevated FGL2 as determined by the method according to claim 1, and
b) treating the subject with an agent that inhibits FGL2.

8. The method according to claim 1 wherein the control FGL2 level is about 30 ng/mL.

9. The method according to claim 1, wherein the level above control is at least 60 ng/mL.

10. The method according to claim 1, wherein the viral hepatitis is mouse hepatitis virus strain 3.

11. The method according to claim 1, wherein the viral hepatitis is chronic hepatitis C.

12. The method according to claim 1, wherein the viral hepatitis is chronic hepatitis B.

13. The method according to claim 1 for determining the prognosis in a subject with viral hepatitis, comprising the steps of:
a) assaying plasma from the subject to determine a level of FGL2; and
b) comparing to a reference level;
wherein a level above the reference level is indicative of poor prognosis.

14. The method according to claim 13, wherein the reference level is 30 ng/mL.

15. The method according to claim 13, wherein the level above the reference level is at least 60 ng/mL.

16. The method according to claim 1, wherein the level of FGL2 is determined with an antibody that binds to FGL2.

17. The method according to claim 7, wherein the subject is treated with an antibody that inhibits FGL2 or by small interfering RNA (siRNA).

18. The method according to claim 16, wherein the antibody is a polyclonal, monoclonal, recombinant or chimeric.

19. The method of claim 16, wherein the antibody is labeled with HRP.

20. The method of claim 1, wherein the level of FGL2 in the plasma sample is determined using an immunoassay.

21. The method of claim 20, wherein the immunoassay is an enzyme-linked immunosorbant assay (ELISA).

22. The method of claim 21, wherein the ELISA is a sandwich type ELISA.

23. An assay useful in the diagnosis of a viral hepatitis comprising the steps of:
a) providing a plasma sample from a subject;
b) reacting the sample with an antibody that binds FGL2;
c) detecting bound FGL2; and
d) comparing the level of FGL2 in the sample with the level of FGL2 in a control subject, wherein a subject having said viral hepatitis is indicated by a greater level of FGL2 in the sample relative to the level of FGL2 in the control subject.

24. The assay of claim 23, wherein the assay is an enzyme-linked immunosorbant assay (ELISA).

25. The assay of claim 24, wherein the ELISA is a sandwich type ELISA.

26. The ELISA of claim 25 comprising a capture antibody and a detector antibody.

27. The ELISA of claim 26 wherein the capture antibody and/or the detector antibody are polyclonal antibodies.

28. The ELISA of claim 26 wherein the capture antibody and/or the detector antibody are monoclonal antibodies.

29. The ELISA of claim 24 for use in the method of claim 1.

30. A kit comprising an antibody that binds FGL2 and instructions for the use thereof in determining the level of FGL2 in a plasma sample.

31. The kit according to claim 30, further comprising a labeled secondary antibody that binds the FGL2 antibody.
